(19) United States
(12) Patent Application Publication
(10) Pub. No.: US 2022/0208396 A1
(43) Pub. Date: Jun. 30, 2022

(54) SYSTEM TO PROVIDE TIMELY PRESCRIPTION INFORMATION

(71) Applicant: RxMedConnet LLC, Dix Hills, NY (US)

(72) Inventors: Brian Bell, Dix Hills, NY (US); Todd Cohen, Dix Hills, NY (US); Steve Williams, Bryantown, MD (US); Brandon Bell, Philadelphia, PA (US)

(21) Appl. No.: 17/645,618

(22) Filed: Dec. 22, 2021

Related U.S. Application Data
(60) Provisional application No. 63/132,604, filed on Dec. 31, 2020.

(51) Int. Cl. G16H 70/40; G16H 10/60

(52) U.S. Cl. CPC G16H 70/40; G16H 10/60

(57) ABSTRACT

Techniques for timely medication information include storing in a database, for each of multiple different medication providers, a pharmacy ID field that uniquely indicates the provider, a pharmacy link field that indicates a network link for the provider, and a pharmacy API format field that indicates a format for requesting patient medication information and for receiving patient medication information from the provider. In response to receiving, from a client process on a remote network node, a client query packet that includes a patient field that indicates information for a patient: selecting a first provider; formatting a pharmacy request packet to request medication information for the patient based on the API format field for the first provider; and, sending the first pharmacy request packet to the network link in the pharmacy link field for the first provider.

### BACKGROUND

A first responder is a person with specialized training who is among the first to arrive and provide care or other assistance at the scene of an emergency, such as an accident, natural disaster, or act of terrorism. First responders typically include law enforcement officers, paramedics, emergency medical technicians (EMTs) and firefighters. In some areas, emergency department personnel are also required to respond to disasters and critical situations, designating them first responders. Currently, a first responder arrives at a scene of an emergency and assesses the human patient(s) and provides care, including determining any medications the patient is currently prescribed. This may occur by observing medication bottles in the vicinity, may be provided by the patient verbally, may be a written list of medications, may be provided by family or friends, or there may be no medication information available.

### SUMMARY

Techniques are provided for augmenting medication information available to first responders, and other urgent or emergency or hospital health care workers, with a system to automatically provide timely medication information based on limited input by the first responders or other health care worker. The system is called herein a first responder prescription (Rx) system, abbreviated FRX hereinafter. However, it is recognized that the system can be used by any other health care workers or clerical workers in a medical setting, such as in an urgent care clinic, emergency room or doctor's office, as well. Applicants have recognized that, without timely information, a patient may be endangered, and patient prospects of recovery diminished, if the first responder or health care worker fails to provide life-saving medications or, conversely, does provide emergency treatment that has harmful interactions with a medication the patient has already taken.

In a first set of embodiments, a method to provide timely medication information, includes storing in a first database, for each of multiple different medication providers, a pharmacy ID field holding data that uniquely indicates the medication provider, a pharmacy link field holding data that indicates a communications network link for the medication provider, and a pharmacy API format field holding data that indicates an application programming interface (API) format for requesting patient medication information and for receiving patient medication information from the medication provider. The method further includes steps taken in response to receiving over a network a first client query packet from a client process on a remote network node, wherein the client query packet includes a patient field that holds data that indicates patent information for a first patient. The steps in response include selecting a first medication provider of the plurality of medication providers. The steps in response also include formatting a first pharmacy request packet to request medication information for the first patient based on the API format field for the first medication provider. Even further in response, the method includes sending the first pharmacy request packet over the network to the communication network link in the pharmacy link field for the first medication provider. In some embodiments of the first set, the method includes storing, in a first patient data record, data that indicates information in the first client query packet.

In some embodiments of the first set, in response to receiving over the network a first pharmacy response packet in response to the first pharmacy request packet, the method even further includes determining whether the first pharmacy response packet indicates the first patient is not among the records at the first medication provider. The method still further includes, if the first pharmacy response indicates the first patient is not among the records of the first medication provider, then selecting a different second medication provider. Under this condition, the method yet further includes: formatting a second pharmacy request packet to request medication information for the first patient based on the API format field for the second medication provider; and, sending the second pharmacy request packet over the network to the communication network link in the pharmacy link field for the second medication provider.

In some embodiments of the first set, in response to receiving over the network a first pharmacy response packet in response to the first pharmacy request packet, the method still further includes retrieving first medication information for the first patient from the first pharmacy response packet based on the API format field for the first medication provider. In response to receiving the first pharmacy response, the method even still further includes: generating a first client response packet that includes the first medication information for the first patient; and, sending the first client response packet over the network to the client process on the remote network node. At the remote network node, data is presented to a user based on the first medication information to enable the user to treat the first patent. In some of these embodiments, the sending the first client response packet is performed within ten minutes of receiving the first client query packet. In some of these embodiments, the method includes storing, in a first patient data record, data that indicates information in the first pharmacy response packet.

In some embodiments of the first set that includes storing, in the first patient data record, data that indicates information in the first client query packet, in response to the first pharmacy response packet, the method further includes retrieving first medication information and independent patient information for the first patient from the first pharmacy response packet based on the API format field for the first medication provider. In response to receiving the first pharmacy response packet, the method yet further includes determining whether the independent patient information is consistent with data already in a first patient data record. Even further still, the method includes, when the independent patient information is consistent, then storing the first medication information and any new independent patient information in the patient data record. Yet more, in response to receiving the first pharmacy response packet, the method includes generating a first client response packet that includes the first medication information for the first patient and any further patient information. Furthermore, in response to receiving the first pharmacy response packet, the method includes sending the first client response packet over the network to the client process on the remote network node, where data is presented to a user based on the first medication information to enable the user to treat the first patient.

In some embodiments of the first set, the “storing in the first database” includes storing in the first database, for each of the medication providers, a pharmacy region field holding data that indicates a geographic region served by the medication provider. The client query packet includes a region field that holds data that indicates a geographic region applicable for the first patient. Then, selecting the first medication includes selecting the first medication provider based at least in part on proximity between the geographic region served by the medication provider and the geographic region applicable for first patient. In some of these embodiments, selecting the first medication provider based at least in part on proximity between the geographic region served by the medication provider and the geographic region applicable for first patient includes expanding a geographic region included in the first pharmacy request packet until a first pharmacy response packet that includes first medication for the first patient is returned in response to sending the first pharmacy request packet. In some of these embodiments, the geographical region applicable for the first patient is a geographical region that includes a home address for the first patient; and, in other of these embodiments, the geographical region applicable for the first patient is a geographical region in a vicinity of the remote network node.

In some embodiments of the first set, the client query packet includes a pharmacy field that holds data that indicates a second medication provider applicable for first patient. In these embodiments, selecting the first medication provider of the plurality of medication providers further comprises selecting the first medication provider based on the second medication provider applicable for first patient.

In a second set of embodiments, a method operating on a client device provides a graphical user interface (GUI) for a user, such as a first responder, to enter what information is available to the user about a patient. That method also generates and sends a first client query packet to a remote server that performs the method according to the first set of embodiments. In some embodiments of the second set, the information for the first client query packet is provided by optical character recognition of a document scanned by the user on site. Upon getting a response, the client device is further configured to present to the user the most comprehensive medication information available. Thereby, the user is enabled to provide proper treatment to the patient.

In a third set of embodiments, a medication GUI presented on a device includes an active area configured to receive patient information, an active area configured to receive medication information, an active area configured to receive location information, and a submit button configured upon activation to cause the device to send a query to a server configured upon receipt of the query to check medication information for the patient over at least one of multiple network links for a corresponding multiple of medication providers.

In other sets of embodiments, a computer-readable medium or a system is configured to perform one or more steps of the above methods or present one or more areas of the above GUI.

Still other aspects, features, and advantages are readily apparent from the following detailed description, simply by illustrating a number of particular embodiments and implementations, including the best mode contemplated for carrying out the invention. Other embodiments are also capable of other and different features and advantages, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

Embodiments are illustrated by way of example, and not by way of limitation, in the figures of the accompanying drawings in which like reference numerals refer to similar elements and in which:

FIG. 1A is a block diagram that illustrates an example cloud-based system 100, according to an embodiment;

FIG. 1B is a block diagram that illustrates example hosts for an FRX system, according to an embodiment;

FIG. 2 is a block diagram that illustrates an example communication data packet for a layer 5 FRX application program, according to an embodiment;

FIG. 3A is a block diagram that illustrates an example FRX query graphical user interface (GUI) for a device operated by user, according to an embodiment;

FIG. 3B is a block diagram that illustrates an example FRX query packet, according to an embodiment;

FIG. 4A is a block diagram that illustrates an example FRX pharmacy data structure, according to an embodiment;

FIG. 4B is a block diagram that illustrates an example FRX patient data structure, according to an embodiment;

FIG. 5 is a block diagram that illustrates an example FRX response packet, according to an embodiment;

FIG. 6A through FIG. 6C are a block diagrams that illustrate example FRX response GUIs for a device operated by a user, according to an embodiment;

FIG. 7 is a flow diagram that illustrates an example method for a FRX client module, according to an embodiment;

FIG. 8 is a flow diagram that illustrates an example method for a FRX server module, according to an embodiment;

FIG. 9 is a block diagram that illustrates a computer system upon which an embodiment of the invention may be implemented;

FIG. 10 illustrates a chip set upon which an embodiment of the invention may be implemented; and

FIG. 11 is a diagram of example components of a mobile terminal (e.g., cell phone handset or tablet) for communications, which is capable of operating in the system of FIG. 1A, according to one embodiment.

### DETAILED DESCRIPTION

A method and apparatus are described for providing timely prescription information to users such as first responders. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Some embodiments of the invention are described below in the context of a first responder user on the scene of some emergency having at least one victim who is a patient. However, the embodiments are not limited to this context. In other embodiments the user is a caregiver or health care worker or intake person at a fixed-site, such as a hospital, nursing home, urgent care center, pharmacy, or emergency room, or the scene is a training exercise and the patient is a real or virtual surrogate human, animal or device, or the user is another person with responsibilities for the patient at the scene or elsewhere. For example, in some embodiments, hospitals use the system to request information from pharmacies about what medications a patient is currently taking or are scheduled to take to ensure that any in-hospital treatment doesn't system to gather data as to whether a person already has had the same or similar prescription filled and active from another pharmacy, in an effort to prevent duplication of medications being dispensed. Such an embodiment provides stricter control and insight into controlled substances.

Techniques for providing best available and timely prescription information to users involve configuring communication network resources to augment automatically what is available in real time on site. As used herein, timely prescription information includes any prescription information for a patient received after a request is sent in one hour or less. In addition, it is successively more advantageous if any prescription information for a patient is received in 30 minutes or less, or in 20 minutes or less, or in near-real time of 10 minutes or less, or in 5 minutes or less, or in 1 minute or less, or in real-time of ten seconds or less, or instantly in one second or less.

Networks of general-purpose computer systems connected by external communication links are well known and widely used in commerce. The networks often include one or more network devices that facilitate the passage of information between the computer systems. A network node is a network device or computer system connected by the communication links. An end node is a node that is configured to originate or terminate communications over the network. An intermediate network node facilitates the passage of data between end nodes.

Communications between nodes are typically effected by exchanging discrete packets of data, called communication packets, or simply “packets,” herein. Information is arranged within packets according to one or more of many well-known, new or still developing protocols. In this context, a protocol consists of a set of rules defining how the nodes interact with each other based on information sent over the communication links. Each packet typically comprises 1] header information associated with a particular protocol, and 2] payload information that follows the header information and contains information that may be processed independently of that particular protocol. In some protocols, the packet includes 3] trailer information following the payload and indicating the end of the payload information. The header includes information such as the source of the packet, its destination, the length of the payload, and other properties used by the protocol. Often, the data in the payload for the particular protocol includes a header and payload for a different protocol associated with a different layer of detail for information exchange. The header for a particular protocol typically indicates a type for the next protocol contained in its payload. The higher layer protocol is said to be encapsulated in the lower layer protocol.

The headers included in a packet traversing multiple heterogeneous networks, such as the Internet, typically include a physical (layer 1) header, a data-link (layer 2) header, an internetwork (layer 3) header and a transport (layer 4) header, as defined by the Open Systems Interconnection (OSI) Reference Model. The OSI Reference Model is generally described in more detail in Section 1.1 of the reference book entitled Interconnections Second Edition, by Radia Perlman, published September 1999, which is hereby incorporated by reference as though fully set forth herein. Data for an application program, such as a webpage or browser, is layer 5 and resides in the payload of the highest layer protocol being utilized. Some protocols pass protocol-related information among two or more network nodes in special control packets that are communicated separately, and which include a payload of information used by the protocol itself rather than a payload of data to be communicated for another protocol or application. These control packets and the processes at network nodes that utilize the control packets are said to be in another dimension, a “control plane,” distinct from the “data plane” dimension that includes the packets with payloads for other applications at the end nodes.

The internetwork header (layer 3) provides information defining the source and destination address within the network. Notably, the path may span multiple physical links. The internetwork header may be formatted according to the Internet Protocol (IP), which specifies IP addresses of both a source and destination node at the end points of the logical path. Internet Protocol version 6 (IPv6), provides for more numerous internet addresses. Thus, the packet may “hop” from node to node along its logical path until it reaches the end node assigned to the destination IP address stored in the packet's internetwork header.

The transport header (layer 4) provides information defining the relationship between one or more packets used for exchanging data between application programs, such as FRX program, executing on the end nodes; and ensures all the data to be transmitted by the application program on one end node is received by the application program other end node. The best-known transport protocol of the Internet Protocol suite is the Transmission Control Protocol (TCP). It is used for connection-oriented transmissions, whereas the connectionless User Datagram Protocol (UDP) is used for simpler messaging transmissions. Together, TCP and UDP comprise essentially all traffic on the internet and are the only protocols implemented in every major operating system.

### 1. FRX System Sructures

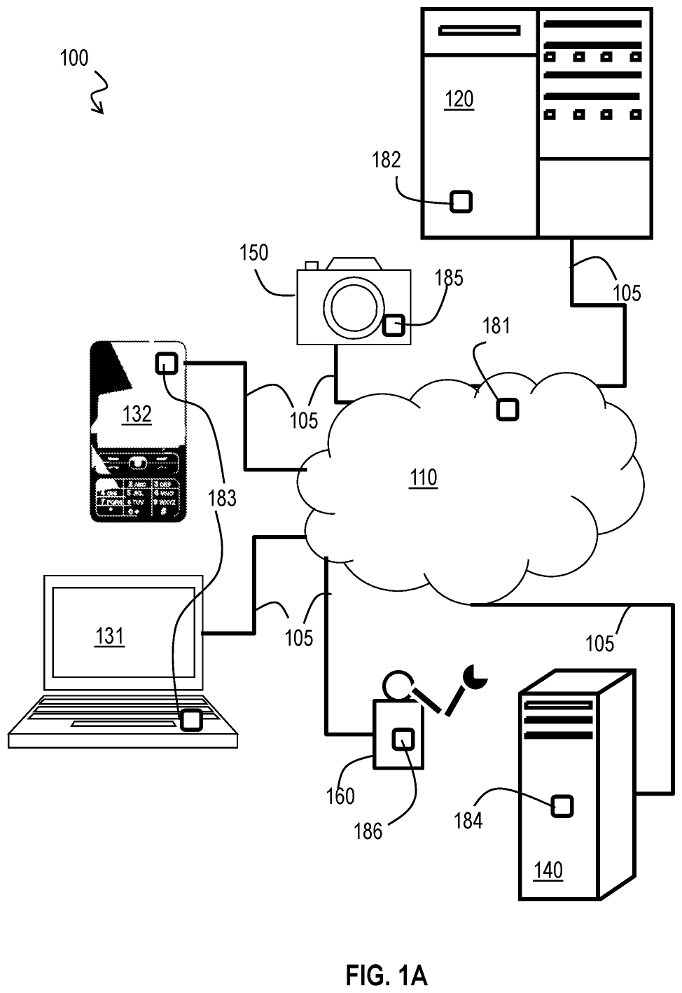

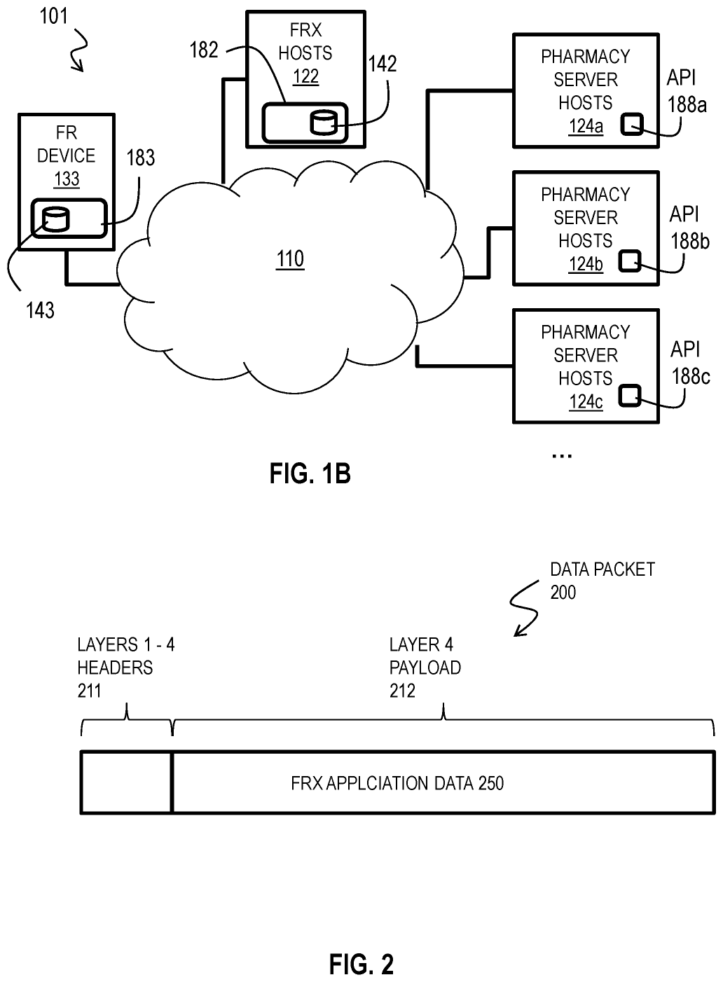

FIG. 1A is a block diagram that illustrates an example cloud-based system 100, according to an embodiment. The system includes a network 110 of interconnected communication devices using wired or wireless single channel or multichannel connections 105. User devices include any electronic device capable of transmitting and receiving signals, such as laptop 131 and mobile device 132 (such as a cell phone, shown, or tablet, not shown). A user device is connected to the network using one or more wired or wireless single channel or multichannel connections 105. One or more server nodes 120, often at different locations, also using one or more connections 105, provide services for devices connected to the system 100, and store data in one or more special storage nodes 140, often at different locations, also using one or more connections 105. In addition, in some systems, one or more sensors 150 (such as digital cameras, telescopes, laser and radar detectors, medical imagers, patient vital signs monitors, etc.) respond to physical phenomena with signals that are converted to data transmitted as packets over connections 105 to other devices in the system 100. In addition, in some systems, one or more actuators 160 (such as assembly line robots, 2D and 3D printers, lasers, radiation sources, etc.) produce physical phenomena in response to signals received as data packets transmitted over connections 105 from other devices in the system 100. Each of these components include one or more software modules that operate the device and communicate with other devices in the systems, as represented by the module 181 for the network 110, FRX module 182 for server(s) 120, FRX module 183 for user device laptop 131 or mobile device 132, module 184 for storage node(s) 140, module 185 for sensor 150, and module 186 for actuator 160. The software modules 181, 182, 183, 184, 185 and 185 are collectively referenced herein as software modules 180.

According to the client-server model, a client process sends a message packet including a request to a server process, and the server process responds by providing a service. The server process may also return a message packet with a response to the client process. Often, but not always, the client process and server process execute on different computer devices, called hosts or nodes, and communicate via a network 110 using one or more protocols for network communications. The term “server” is conventionally used to refer to the process that provides the service, or the host computer on which the process operates. Similarly, the term “client” is conventionally used to refer to the process that makes the request, or the host computer on which the process operates. As used herein, the terms “client” and “server” refer to the processes, rather than the host computers, unless otherwise clear from the context. In addition, the process performed by a server can be broken up to run as multiple processes on multiple hosts (sometimes called tiers) for reasons that include reliability, scalability, and redundancy, but not limited to those reasons.

In the illustrated embodiment, the FRX system includes an FRX client 183 and an FRX server 182, each on one or more hosts in a dedicated or public communications network 110, such as the internet. Furthermore, the FRX system makes use of two or more pharmacy server hosts. FIG. 1B is a block diagram that illustrates example hosts for an FRX system 101, according to an embodiment. A first responder (FR) device 133 is a laptop, cellphone, tablet or other data processing device configured for network communications and in the possession of the user, such as a first responder. The FR device 133 executes a FRX client module 183 which may also use a FRX client data structure 143, such as a file or database. Available through network 110 are one or more FRX hosts 122 for an FRX server module 182 that includes one or more FRX server data structures 142.

Also available through network 110 are two or more pharmacy server hosts 124a, 124b, 124c, among others indicated by ellipsis, collectively referenced as pharmacy hosts 124. Each pharmacy host 124 is equipped with a module (not shown) that manages the patients and medication information for a particular pharmacy or chain of pharmacies or other provider of medication for patients, such as a health maintenance organization (HMO), hospitals, or a government agency like the Veterans Administration (VA). As used herein, a medication provider means any entity that maintains or stores data about any prescription for a patient. Access to that information at each pharmacy host 124 is through a corresponding application programming interface (API), such as API 188a, 188b, 188c, among others, collectively referenced hereinafter as pharmacy API 188. Each pharmacy API 188 (e.g., API 188a) is particular for its corresponding pharmacy host 124 (e.g., 124a), and may be different than the API (e.g., API 124b) for a different pharmacy host (e.g., 124b).

A function of the FRX client 183 is to request medication information for a patient in the vicinity of the FR device 133 and to receive, and present to a first responder or other user, the medication information requested. In response, a first responder or other health care worker can provide proper treatment to the patient by administering life-saving medication or by avoiding administering other medications that may have deleterious interactions with medications the patient has taken or is taking.

A function of the FRX server 182 is to provide a central site for translating requests from FRX client 183 to requests for the pharmacy API 188 of one or more pharmacies, to receive the information from the pharmacy API 188, and to send the medication information back to the FRX client 183 in a timely way. As used herein, a response sent to the FRX client is timely if it is sent within a time after the request from the FRX client that is in a range from 1 second to 100 minutes, or a range from 10 seconds to 100 minutes, or in a range from 1 minute to 100 minutes, or in a range from 1 second to 10 minutes. The shorter the time to respond, the more advantageous for the proper treatment of the patient.

Although processes, equipment, and data structures are depicted in FIG. 1A and FIG. 1B as integral blocks in a particular arrangement for purposes of illustration, in other embodiments one or more processes or data structures, or portions thereof, are arranged in a different manner, on the same or different hosts, in one or more databases, or are omitted, or one or more different processes or data structures are included on the same or different hosts.

The FRX client 183 and FRX server 182 communicate using FRX data packets. FIG. 2 is a block diagram that illustrates an example communication data packet 200 for a layer 5 FRX application program, according to an embodiment. In other embodiments, the FRS application date may be sent in other protocol layers, such as UDP in layer 4. FIG. 2 illustrates an example data packet 200 with data provided for a Layer 5 or higher layer protocol. Such data packets 200 include headers 211 made up of a series of bits that indicate values for various Layer 1 through Layer 4 header fields, and a Layer 4 payload 212 that has the FRX application (layer 5) data. For example, the Layer 3 and Layer 4 header fields destination and port data indicate the FRX host 122 IP address and FRX server 182 on that host, respectively. In some embodiments the FRX data is provided within another layer 5 protocol, such as HTTP for a World Wide Web browser and web server.

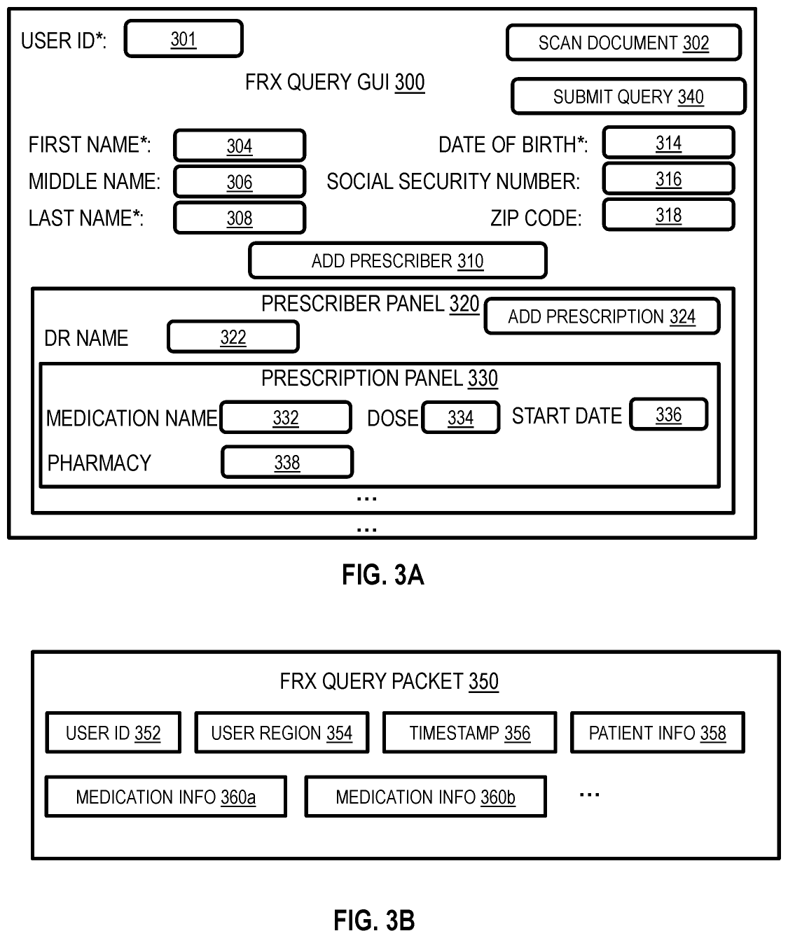

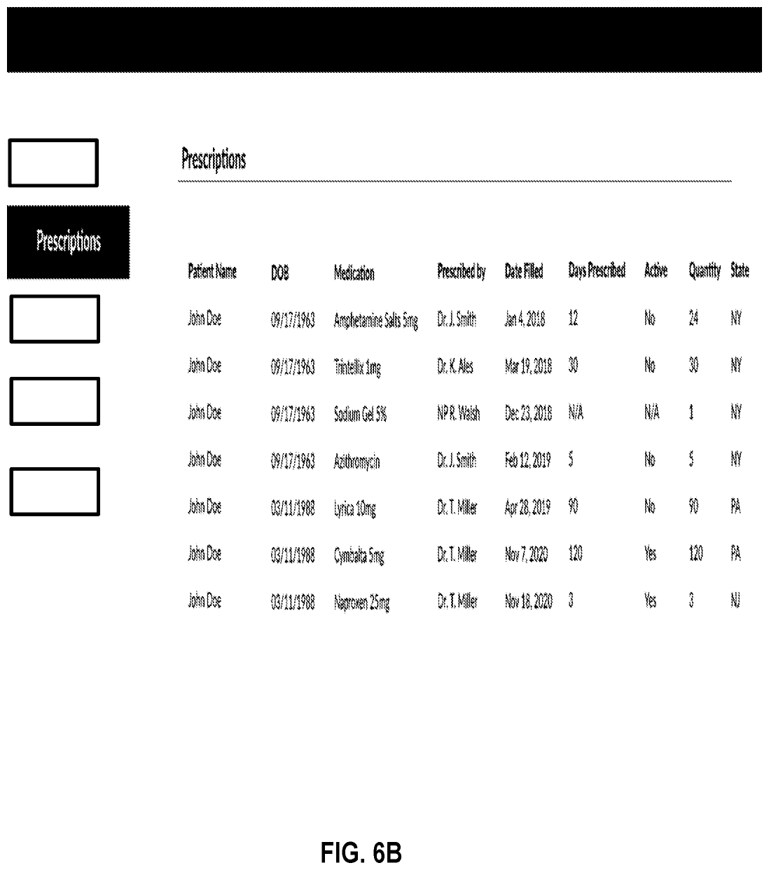

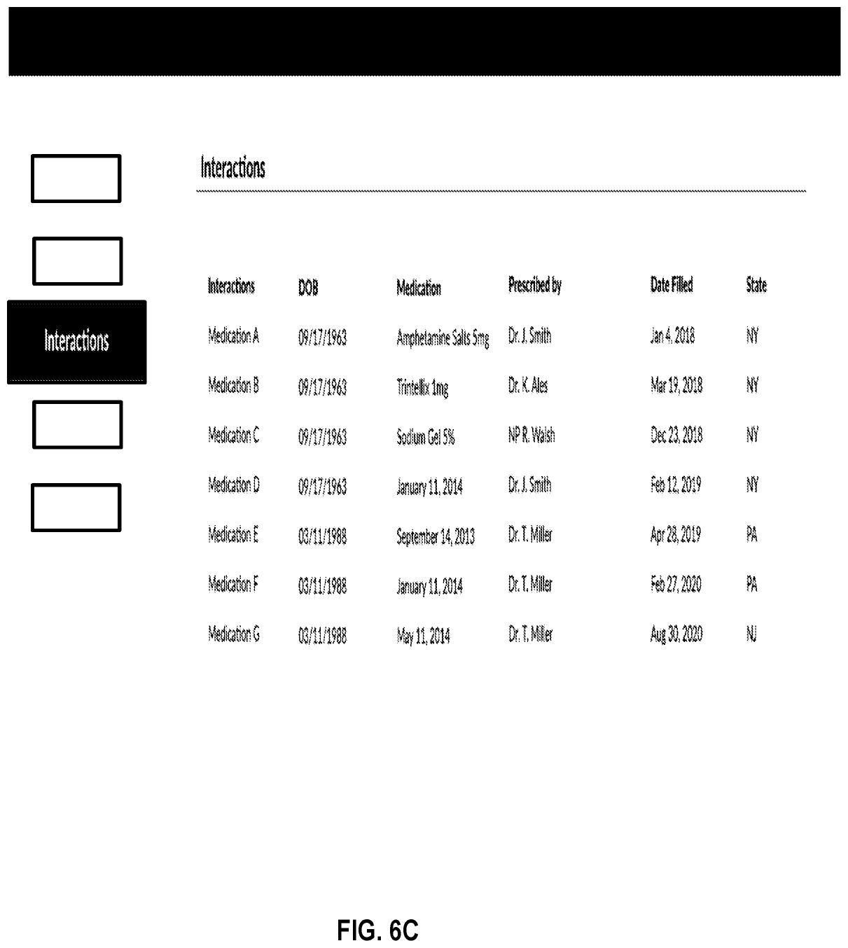

FIG. 3A is a block diagram that illustrates an example FRX query graphical user interface (GUI) 300 for a device operated by a user, according to an embodiment. Thus, FIG. 3A is a diagram that illustrates an example screen on a display device of FR device 133. In any GUI, the screen includes one or more active areas that allow a user to input data to operate the client. As is well known, an active area is a portion of a display to which a user can point using a pointing device (such as a cursor and cursor movement device, or a touch screen) to cause an action to be initiated by the device that includes the display. Well known forms of active areas are standalone buttons, radio buttons, check lists, pull down menus, scrolling lists, and text boxes, among others. Although areas, active areas, windows and toolbars are depicted in FIG. 3A, and a later GUI in FIG. 6, as integral blocks in a particular arrangement on particular screens for purposes of illustration, in other embodiments, one or more screens, windows or active areas, or portions thereof, are arranged in a different order, are of different types, or one or more are omitted, or additional areas are included or the GUI is changed in some combination of ways. In some embodiments, one or more of the steps associated with these active areas are programmed manually or are learned using artificial intelligence (AI) or machine learning (ML), including deep ML, or some combination. In some embodiments, the AI/ML is trained and maintained on the FRX server 182 and performs its functions there and returns any results to the FRX client 183. In some embodiments, previously trained AI/ML components are imported into the FRX client 183 and perform their functions within the FRX client 183.

In the illustrated embodiment, the GUI includes an active area 301 labelled “user ID*.” An asterisk in a label indicates the associated active area requires some user input by the user, in the illustrated embodiment. The user selects this area to identify the user, e.g., by typing a username or identifier into a text box active area. In some embodiments, selecting this active area also involves entering a password or other authentication mode. In some embodiments, the device 133 is equipped with a sensor 150, suitable for detecting a voice print or spoken command, or fingerprint, or a retina or iris or facial or bar code image and active area 131 activates the sensor and performs the recognition function to complete the authentication of the user using the associated authentication mode.

The query GUI also includes active areas suitable for the user to provide patient information for use in uniquely identifying the patient. In the illustrated embodiment, the query GUI 300 includes active area 304 labeled “First name*”, active area 306 labelled “Middle name,” active area 308 labelled “Last name*,” active area 314 labeled “Date of Birth*,” active area 316 labelled “Social security number,” and active area 318 labelled “Zip code.” In other embodiments more or fewer active areas are included; for example, in some embodiments an active area (not shown) labeled “telephone number” is included. Active areas 304 through 308 are well suited for text boxes, which when selected present a keyboard allowing a user to type in text, or a microphone icon indicating a voice recognition system allows the user to speak the names and or spellings thereof. Active area 314 is well suited for a pop-up calendar. Active area 316 can be a text box, as described above or one already formatted for a social security number, with dashes and limits on how many digits can be entered beside each dash. Active area 318 can be a list that can be scrolled, or the list reduced to entries consistent with the digits the user has already typed or recited, or some combination. In some embodiments, the active area displays not only the zip code entered, but also the region (e.g., city or county or state) associated with the entered code to help make evident any typographical errors. The zip code entered is one associated with the patient, such as the patient's residence. In some embodiments, to cover patients who reside in another country, a country active area is added and the zip code is used for any province or province code used in that country.

A submit query active area 340, such as a button, or voice activated sensor, causes the FRX client 183 to generate, and send to the FRX server 182, a query data packet with data indicating the contents of any or all of active areas 304, 306, 308, 314, 316 and 318, as described in more detail below with reference to FIG. 3B.

It is anticipated that, in an emergency situation, not all of the patient information will be available. In such circumstances, the information available might not be sufficient to enable uniquely identifying the patient. In such circumstances the GUI is configured to repeat a request for a required active area to be used. However, if all required areas are not filled, then the query can still be submitted by the user and the remote FRX server 182 is tasked with a probabilistic search for the patient's medication information, e.g., using AI or ML.

The query GUI 300 also includes active areas suitable for the user to provide any patient medication information available at the scene. Such information may be available from the patient, or from one or more human companions to the patient, or from a document listing several medications, or from a container of each of one or more prescription medications available at the scene or a building near the scene. Because prescription medications are prescribed by a person with authority to do so, the medication information includes any information available about the prescribing person or persons (such as one or more doctors or physician assistants or other technicians or some combination, each called a prescriber, herein). Such information may also be helpful in distinguishing between patients with the same name or other patient information, a process called herein disambiguating the patient. For example, if date of birth is not known, the prescriber name may be able to distinguish a patient from a nephew with the same name by giving an approximate age for the patient, unless the nephew is too old to be distinguished in age from the patient. In some embodiments, AI/ML is employed to disambiguate the patient.

In the illustrated embodiment, the query GUI 300 includes active area 310, such as a button, configured to add a prescriber for the patient. Each time the add prescriber active area 310 is activated by the user, the FRX client is configured to present a new prescriber panel 320 on the GUI 300, on the same screen or a new screen or some combination. Any additional prescriber panel in the GUI 300 is indicated by ellipsis outside the illustrated prescriber panel 320. The prescriber panel 320 includes active area 322 labelled “Dr. Name” and active area 320 for adding a prescription for the current prescriber. A default prescriber name is “unknown” or similar designation; and can be used for all medications entered at the scene for which the prescriber is not known. In some embodiments, the active area 322 is a text box. In some embodiments, the active area is a pull-down menu with one menu item selected for the user to manually key in a prescriber name, a second menu item to speak the prescriber's name using voice recognition, and another choice for scanning a document that includes the prescriber name, such as a portion of a prescription bottle.

In the illustrated embodiment, the prescriber panel 320 includes active area 324, such as a button, configured to add an on-scene discovered prescription for the patient. Each time the add prescription active area 324 is activated by the user, the FRX client is configured to present a new prescription panel 330 on the GUI 300, such as within the prescriber panel 320, as illustrated, on the same screen or a new screen or some combination. Any additional prescription panel in the GUI 300 is indicated by ellipsis outside the illustrated prescription panel 330 and inside the illustrated prescriber panel 320. The prescription panel 330 includes active areas 322, 334, 336 and 338 labelled “Medication name,” “Dose,” “Start date,” and “Pharmacy,” respectively. In some embodiments, each of the medication active area 332 and pharmacy active area 338 is a text box. In some embodiments, the active area is a pull-down menu with one menu item selected for the user to manually key in a name, a second menu item to speak the name using voice recognition, and another choice for scanning a document that includes the name, such as a prescription bottle. In some embodiments using a text box, the active area is a pop-up list that can be scrolled, or the list is reduced to entries consistent with the characters the user has already typed or recited. The dose active area 334 can be a numeric field with a list or pull-down menu for selecting units (e.g., milligrams, mg, or teaspoonfuls, tsp). In some embodiments, the dose active area 334 or prescription panel also includes one or more active areas to enter a dose schedule indicating how often the dose is to be taken, e.g., four times daily for ten days. Start date active area 336 is well suited for a pop-up calendar. In some embodiments, one or more fields or active areas are filled automatically. For example, AI/ML can determine from data already entered what condition the patient likely has and what medications are typically prescribed together for that condition and use that information to prefill one or more prescription fields, which the user can accept or delete. In some embodiments, AI/ML is used to flag any harmful interactions among the active/recently dispensed medications.

In some embodiments, the FRX Query GUI 300 includes a scan document active area 302, such as a button. When scan document active area 302 is activated, a sensor 150 connected to, or integrated in, the client device 133 scans a document provided and the FRX client is further configured to automatically detect the information for, and populate, one or more areas of the GUI 300. For example, an optical scanner scans the document, performs optical character recognition, and uses natural language processing tools or other machine learning (AI/ML) tools to automatically detect the information for, and populate, one or more areas of the GUI 300. In other embodiments, the sensor 150 is any machine scanner, such as magnetic strip reader, or radio frequency identification (RFID) chip reader, or bar-code scanner, or Q-code scanner, or other sensor, or some combination. For example, a driver's license or other ID card of the patient is machine scanned and the patient name and zip code are automatically determined and used to populate one or more of active areas 304, 306, 308 and 318. As another example, a medication container is machine scanned and a patient name, prescriber name, prescription name, dose amount, dose schedule, start date or pharmacy name, or some combination, is determined automatically and used to populate one or more active areas 304, 308, 322, 332, 334, 336 and 338.

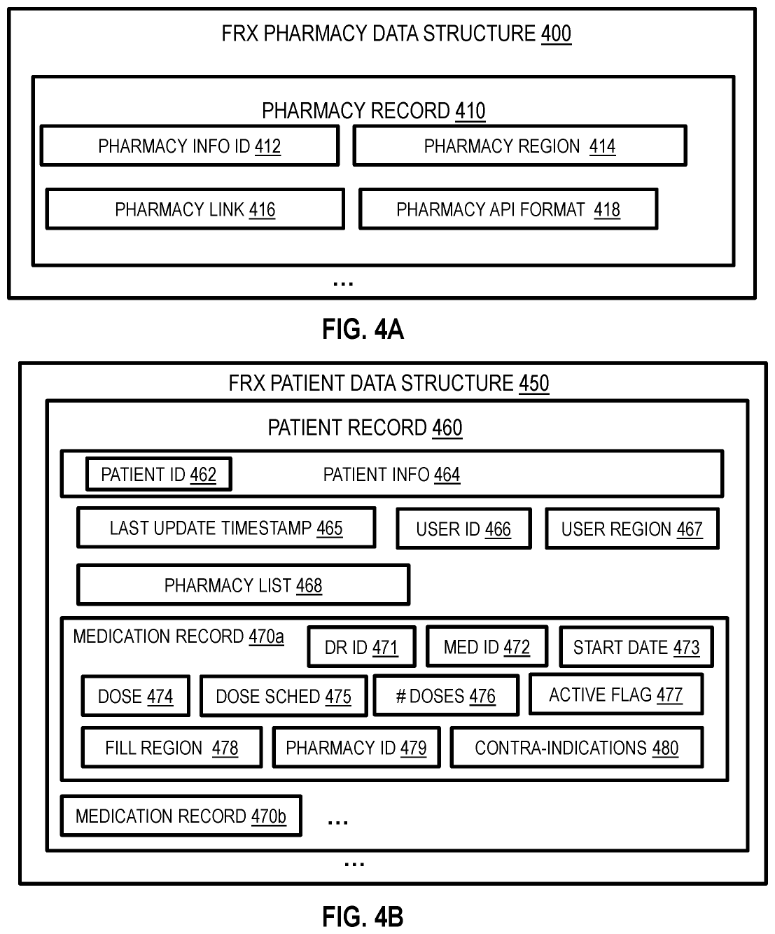

In some embodiments, the information obtained through operating GUI 300 is stored by the FRX client 183 locally in a patient data structure 143. FIG. 4B, described in more detail below, depicts a patient data structure used at an FRX server, which data structure can be used locally, in whole or in part, as patient data structure 143, in some embodiments.

As mentioned above, when a user activates the submit query active area 340, the FRX client 183 generates, and sends to the FRX server 182, one or more query data packets with data indicating the contents of any or all of active areas of the GUI 300. FIG. 3B is a block diagram that illustrates an example FRX query packet 350, according to an embodiment. The data query packet 350 includes, in a layer 4 payload 212, FRX application data 250 including one or more of a user identifier (ID) field 352, a user region field 354, a timestamp field 356, a patient information field 358, and zero or more medication information fields 360a, 360b among others indicated by ellipsis, collectively referenced hereinafter as medication information fields 360. In some embodiments that use multiple data query packets to convey all this information, the user ID field 352 or the patient information field 358, or some portion, or some combination, can serve as a FRX header field that is repeated in each packet of the set of packets. In various embodiments, the data in each field of data query packet 350 can be compressed or encrypted or otherwise formatted differently than in the query GUI 300 or data structure 143. In some embodiments, such formatting is implemented to comply with all privacy requirements such as HIPAA and NIST.

The user ID field 352 holds data that indicates a unique identifier for the user, based at least in part on information provided in active area 301. The user region 354 holds data that indicates a region where the user is operating the FR device 133, which can be determined based on a table stored for the user in data structure 143, such as the home base for the user, or determined from a global positioning system (GPS) or other geolocation system, based on measurements by a geolocation sensor 150 attached to, or integrated in, the device 133. The timestamp field 356 holds data that indicates an absolute time that the query packet was generated at the FRX client 183; and, serves to distinguish one query packet 350 from another by the same user as well as to set a time window for the FRX server to provide a response.

Patient information field 358 holds data that indicates the patient information captured at the query GUI 300, e.g., in active areas 304, 306, 308, 314, 316, or 318, or some combination. Each medication information field 360 holds data that indicates the medication information, if any, captured at the query GUI 300, e.g., in active areas 322, 332, 334, 336, or 338, or some combination, for one prescription panel 330.

The FRX server 182 is configured to maintain one or more data structures 142 to respond to a query from the FRX client 183, in various embodiments. FIG. 4A is a block diagram that illustrates an example FRX pharmacy data structure 400, according to an embodiment. Data structure 400 is an example embodiment of one of the data structures 142 maintained by the FRX server 182. Data structure 400 includes multiple pharmacy records 410, one for each medication provider organization, such as a pharmacy chain or small business pharmacy, hospital, HMO, or government agency, known or discovered by the FRX server over time. Other pharmacy records are indicated by ellipsis. Discovery of medication providers can be performed in any manner known, such as Web crawl programs, or AI/ML approaches, among others, alone or in combination.

In each pharmacy record 410, the pharmacy information and identifier (ID) field 412 holds data that uniquely indicates one of the known medication provider organizations. In various embodiments this field 412 holds data that indicates the medication provider name, address, telephone number, and a unique number, called a pharmacy ID, assigned by the FRX server, or some other authority, that uniquely identifies one medication provider and can be used as a key in a database. The pharmacy region field 414 holds data that indicates one or more regions where the organization indicated in field 412 provides pharmacy services. For example, in some embodiments, field 414 indicates a list of zip codes, or cities, or states, or some combination, where the organization provides prescription medications.

The pharmacy link field 416 holds data that indicates a network communications address, where patient prescription information can be obtained. For some smaller medication provider organizations, the address is simply a telephone number that involves a user calling a facility run by the organization to verbally interact with a human or robotic agent of the organization. More commonly, and more usefully, the address is a network link, such as a universal resource locator (URL), or an internet address and application layer port, for an API 188 at a pharmacy server host 124 configured to receive and process requests for patient prescription information.

The pharmacy API format field 418 holds data that indicates an order and format, (including any units, value offsets and scaling factors and null values), for inputting information (such as user authorization, patient name, birthdate, SSN, zip code, prescriber name, or pharmacy location, or some combination) to the API 188. The pharmacy API format field 418 also holds data that indicates an order and format of data returned through the API 188 from the pharmacy host 124, such as values for patient information missing in the input, the prescription name, dose, schedule, start date, prescribing prescriber, store location, whether the prescription is active or expired, or some combination. The pharmacy API format field 418 is null or blank for medication provider organizations that do not provide an API.

In some embodiments, the FRX server 182 accumulates patient prescription information over time from several pharmacy APIs 188 and stores this accumulated knowledge in a FRX patient data structure as part of data structure 142. FIG. 4B is a block diagram that illustrates an example FRX patient data structure 450, according to an embodiment. Data structure 450 is an example embodiment of one of the data structures 142 maintained by the FRX server 182. Data structure 450 includes multiple patient records 460, one for each patient for whom requests are, or recently had been, pending. Other patient records are indicated by ellipsis outside patient record 460. In some embodiments, patient records are purged after an appropriate time, such as 24 hours or 30 days or after handoff to a facility that takes over responsibility for the patient. In some embodiments, such purging is implemented to comply with all privacy requirements such as HIPAA and NIST.

In the illustrated embodiment, each patient record 460 includes a patient ID field 462, a patient information field 464, a last update timestamp field 466, a user ID field 352, a user region fields 354, a pharmacy list field 468, and zero or more medication records 470a, 470b, among other indicated by ellipsis inside patient record 460, collectively referenced as medication records 470. The patient ID filed 462 holds data that uniquely indicates a particular patient, such as a series number assigned by the FRX server 182 upon receiving a first query for the patient, or a combination user ID and timestamp in fields 352 and 356 of the first FRX query packet. The patient information field 464 holds data that indicates the patient information known about this patient, such as any information in one or more FRX queries, augmented by any patient information provided in an API response from a pharmacy host, including any information about patient name, birthdate, SSN, zip code, home street address, secondary address, phone number(s), emergency contact, prescribers, etc.. The last update timestamp field 465 holds data that indicates a time when the patient record was most recently updated, whether based on receipt of a RFX query packet or receipt of a response from a pharmacy host through its API 188. The user ID field 466 and user region field 467 hold data based on data in fields 352 and 354, respectively, of the most recently received FRX query packet 350; and, in some embodiments the data is identical. The pharmacy list field 468 holds data that indicates zero or more medication providers that have provided information about the patient indicated in field 462, e.g., through an API 188. In some embodiments, the pharmacy is identified in the list using the data in the pharmacy ID field 412 of the FRX pharmacy data structure 400. Such a list as in field 468 is handy if, subsequently, an update is requested for medications for this particular patient.

In the illustrated embodiment, each medication record 470 includes a prescriber identification (DR ID) field 471, a medication identification (MED ID) field 472, a start date field 473, a dose field 474, a dose schedule field 475, a number of doses (# doses) field 476, an active flag 477, a fill region field 478, a pharmacy ID field 479, and a contra-indications field 480. The DR ID field 471 holds data that identifies a prescriber who prescribed the medication, as uniquely as possible, e.g., by first and last name, practice address(s), license to practice with date and licensing authority, or some combination. If the prescriber is not known, then field 471 holds a default value indicating unknown prescriber.

The MED ID field holds data that uniquely indicates the medication, using its brand name, generic name, or health system code and code supplying authority, or some combination. The start date field 473 holds data that indicates the start date of the most recent prescription for which information has been received by the host maintaining the data structure 450 (e.g., FRX server 182 in data structure 142 or FRX client 183 in data structure 143). Similarly, the dose field 475, dose schedule field 475 and # doses field 476 hold data describing each of those properties, respectively, for the most recent prescription for which information has been received. Each holds a null or default value if no corresponding information has been received. The active flag 477 holds data that indicates whether the prescription described in fields 473 through 476 is past its expiration date. In some embodiments, the active flag is a single bit with one value indicating active and the other value indicating inactive, with a null value being the default if no information has been received. In some embodiments, the active flag 477 includes data that indicates the expiration date or a null value if no information has been received.

The fill region field 478 holds data that indicates a geographic region served by the medication provider, such as by a single branch of a pharmacy chain, and can be indicated in any manner, such as a city, county, zip code or state/province. The pharmacy ID field holds data based on the pharmacy ID field in the pharmacy info ID field 412 in the FRX pharmacy data structure 400 for the medication provider that filled the prescription indicated in the present record 470. The contra-indications field 480 holds data that indicates any warning for the use of the medication indicated in field 472, such as patient vital signs condition or other medications that, when combined with administering the medication indicated in field 472, can cause harm to the patient. This information may be based on information printed on the prescription container or looked up manually or automatically in some medical reference, e.g., using AI/ML.

In some embodiments, some patient record fields are maintained indefinitely, in case of future emergencies. In some embodiments, records are purged on a regular schedule or after a certain time, such as 24 hours or 30 days, after last being updated. In some embodiments, one or more fields have individual expiration dates indicating when those fields are purged individually. In some embodiments, such purging is implemented to comply with all privacy requirements such as HIPAA and NIST.

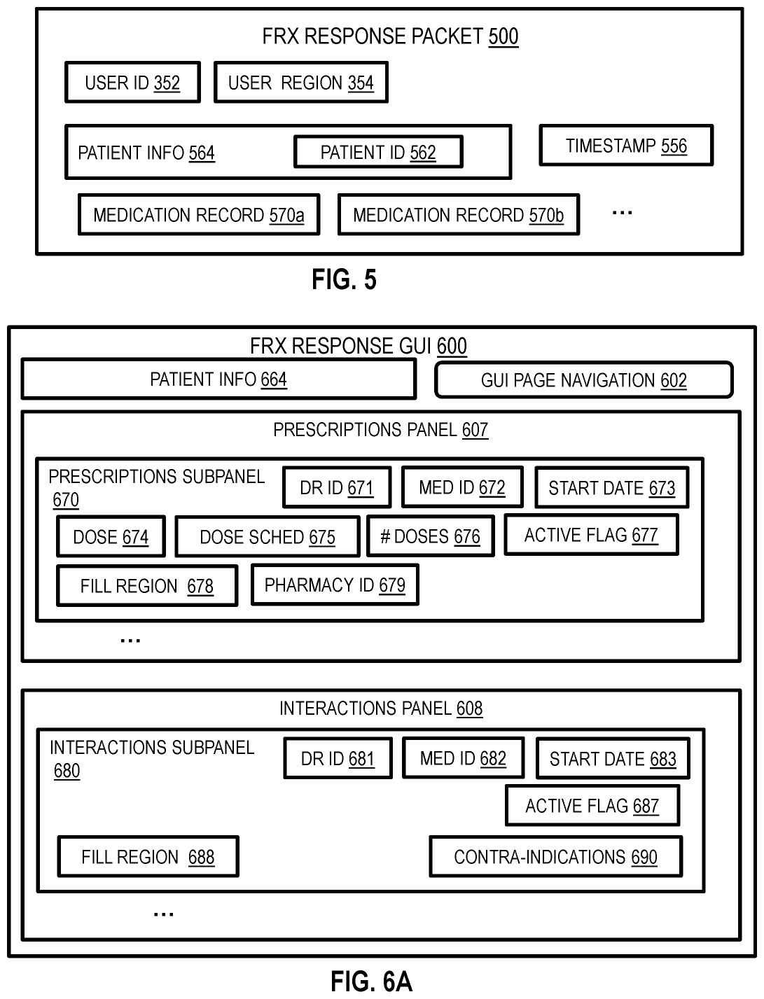

In some timely way after receiving a FRX query packet or new prescription information through some pharmacy API 188 that causes an update to patient record 460, a FRX response packet with all known medication information is sent from the FRX server to the FRX client that sent the query packet. In some embodiments, a response packet is sent every time the patient data structure is updated. In some embodiments, a response packet is sent no more frequently than a certain refresh time in a range from 1 minute to 10 minutes after the query or the last response. FIG. 5 is a block diagram that illustrates an example FRX response packet 500, according to an embodiment.

The FRX response packet 500 includes, in a layer 4 payload 212, FRX application data 250 including, in the illustrated embodiment, one or more of a user identifier (ID) field 352, a user region field 354, a patient ID field 562 within a patient information field 564, a timestamp field 556, and zero or more medication information fields 570a, 570b among others indicated by ellipsis, collectively referenced hereinafter as medication information fields 570. In some embodiments that use multiple data query packets to convey all this information, the user ID field 352 or the patient ID field 562 or the patient information field 564, or some portion, or some combination, can serve as a FRX header field that is repeated in each packet of the set of packets. In various embodiments, the data in each field of FRX response packet 500 can be compressed or encrypted or otherwise formatted differently than in the query GUI 300 or data structure 142 including data structure 450. In some embodiments using multiple query response packets, a last data packet is sent to indicate no further query response packets are being sent and the communication is complete. In some embodiments, this completed transmission condition is indicated by the layer 3 protocol, such as UDP or TCP In some embodiments, such formatting is implemented to comply with all privacy requirements such as HIPAA and NIST.

The user ID field 352 and user region 354 holds data as indicated above for the FRX query packet 350. The patient ID field 562 holds data based on field 462 that uniquely indicates the patient record 460 in the FRX patient database 400 maintained by the FRX server 182 in data structure 142. Use of this ID can benefit further communications between the FRX client 183 and the FRX server 182. The timestamp field 356 holds data that indicates an absolute time that the patient record 460 was last updated and is based on data in field 465 of the FRX data structure 450 maintained by the FRX server 182. Patient information field 564 holds data that indicates some or all of the patient information in field 464 of the patient record 460 maintained by the FRX server 182 in data structure 142. Thus, the information indicated by the data in field 564 is possibly more up to date and more complete than patient information in field 358 of the previous FRX query packet 350. Each medication information field 570 holds data that indicates the medication information, if any, in one medication record 470 of the patient record 460 for the current patient maintained by the FRX server 182 in data structure 142. Thus, the information indicated by the data in medication fields 570 is possibly more up to date and more complete than medication information in fields 360 of the previous FRX query packet 350.

Upon receipt of FRX response packet 500, the FRX client 183 presents information to a user in a FRX response GUI and, in some embodiments, stores the information in local data structure 143 using an arrangement such as described in FIG. 4B for the FRX patient data structure 460. FIG. 6A is a block diagram that illustrates an example FRX response GUI for a device operated by a user, according to an embodiment. In the illustrated embodiment, the FRX response GUI 600 includes a patient information area 664 that displays at least some of the information received in field 564 of the most recent FRX response packet 500 from the FRX server 182 (e.g., as determined using the time indicated in timestamp field 556 of that response). In some embodiments, area 664 is an active area that is configured to expand and present more detailed patient information when activated.

In the illustrated embodiment, the FRX response GUI 600 includes a prescriptions panel 607. The prescriptions panel 607 includes zero or more prescription subpanels 670, with any other subpanels indicate by ellipsis inside the prescription panel 607. In the illustrated embodiment, the FRX response GUI 600 also includes an interactions panel 608. The interactions panel 608 includes zero or more interactions subpanels 680, with any other subpanels indicate by ellipsis inside the interactions panel 608. In the illustrated embodiment, the FRX response GUI 600 also includes a GUI page navigation active area 602 configured to allow the user to move through the various panels that might not fit on one screen of the display device or to drill down on any presentation area to obtain more detail about that presentation.

Each prescription subpanel displays at least some of the information in the most recent fields 570 of any FRX response packet 500 received from the FRX server 182. For example, DR ID area 671, MED ID area 672, start date area 673, dose area 674, dose schedule area 675, # doses area 676, active flag area 677, fill region area 678 and pharmacy ID area 679 each presents at least some information based on the most recent data sent by the FRX server from fields 471, 472, 473, 474, 475, 476, 477, 478 and 479, respectively, in the FRX server patient data structure 450 for the particular patient. In some embodiments, each of one or more of areas 671, 672, 673, 674, 675, 676, 677, 678 and 679 is an active area that is configured to expand and present more detailed information when activated. FIG. 6B is a block diagram that illustrates an example prescription panel. The boxes along the left edge are within an active area 602 for navigating between screens and has been activated to display the prescriptions panel for two current patient(s), e.g., a father and son with the same name but different birthdates. Seven prescription subpanels are displayed each presenting patient name and date of birth for the patient information area 664, medication name from the MED ID area 672, prescriber name from the DR ID area 671, date filled from the start date area 673, days prescribed from the dose schedule area 675, active status from the active flag area 677, quantity from the # doses area 676 and state from the fill region area 678.

Each interactions subpanel displays at least some of the information from the contra-indications portion in the most recent fields 570 of any FRX response packet 500 received from the FRX server 182. For example, DR ID area 681, MED ID area 682, start date area 683, active flag area 687, fill region area 688 and contra-indications area 690 each presents at least some information based on the most recent data sent by the FRX server from fields 471, 472, 473, 477, 478 and 480, respectively, in the FRX server patient data structure 450 for the particular patient. In some embodiments, each of one or more of areas 681, 682, 683, 687, 688 and 690 is an active area that is configured to expand and present more detailed information when activated. FIG. 6C is a block diagram that illustrates an example interactions panel. The boxes along the left edge are within the active area 602 for navigating between screens and has been activated to display the interactions panel for two current patient(s). Seven prescription subpanels are displayed each presenting interacting medications from the contra-indications area 690, patient date of birth from the patient information area 664, prescribed medication from the MED ID area 682, prescriber from the DR ID area 681, date filled from the start date area 683, and state from the fill region area 688.

Although data structures, messages and fields are depicted in FIG. 2 through FIG. 6C as integral blocks in a particular order for purposes of illustration, in other embodiments, one or more data structures or messages or fields, or portions thereof, are arranged in a different order, in the same or different number of data structures or databases in one or more hosts or messages, or are omitted, or one or more additional fields are included, or the data structures and messages are changed in some combination of ways.

### 2. FRX System Methods

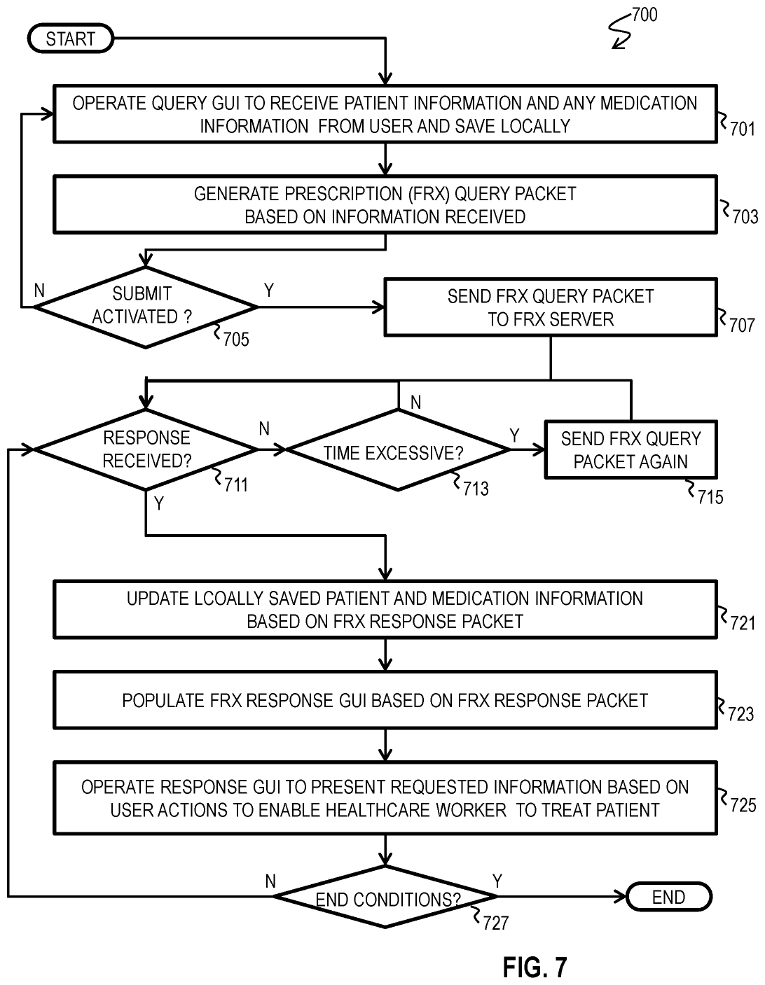

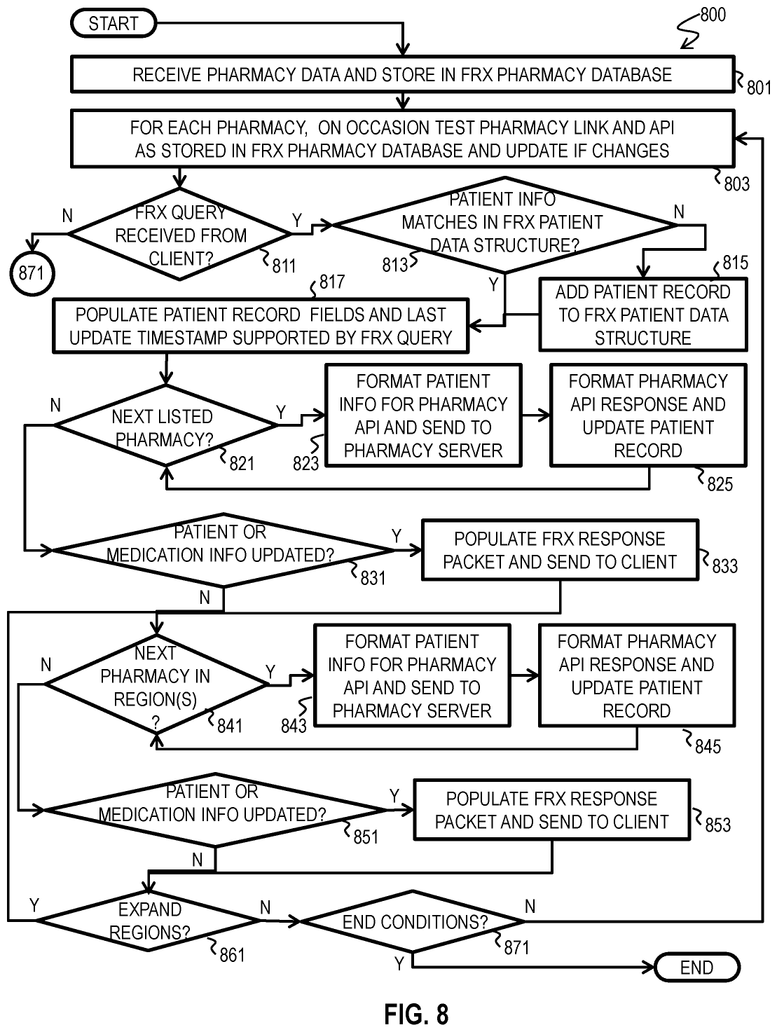

FIG. 7 is a flow diagram that illustrates an example method 700 for a FRX client module 183, according to an embodiment. Although steps are depicted in FIG. 7, and in subsequent flowchart FIG. 8, as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways.

In step 701, the FRX client 183 presents a FRX query GUI (e.g., FRX query GUI 300 depicted in FIG. 3A) and operates in response to any inputs by a user to receive any available patient information and any medication information from a user, such as a first responder. The information received is saved locally, at least in an image of the GUI 300. In some embodiments the information is also stored in a buffer in which a FRX query packet (e.g., FRX query packet 350 depicted in FIG. 3B) is being generated. In some embodiments the information is also stored locally in a local patient data structure 142 similar to the FRX patient data structure 450 formed at the FRX server 182 and depicted in FIG. 4B. In some embodiments, step 701 includes operating any sensor 150 to authenticate a user, such as a first responder (e.g., based on active area 301), and to scan any documents, such as a patient ID card or a prescription container for the patient (e.g., based on active area 302).

In step 703 a FRX query packet is generated or updated in a local buffer based on the information received through the FRX query GUI. This includes generating the Layer 1 through Layer 4 headers for the data packet to navigate the network 110 and arrive at the FRX server 182. This also includes translating, compressing or encrypting any information as desired before transmitting over the network 110. In some embodiments, such formatting is implemented to comply with all privacy requirements such as HIPAA and NIST.

In step 705, it is determined if a submit query active area (e.g., active area 340) of the RTX query GUI has been activated. If not, control passes back to step 701 to continue to operate the GUI. If the submit active area has been activated, then control passes to step 707 and following, described below. In some embodiments, when the submit active area is activated, step 705 includes checking to make sure any required fields have been filled. If not, control returns again to step 701 with a message to fill in required fields. If the submit active area is activated again after that message is sent, then control passes to step 707 and following to proceed with the sparse information that is available.

In step 707, a current time is used to fill the timestamp field 356 in a FRX query packet (e.g., packet 350), and the FRX query packet is sent over network 110 to FRX server 182.

In step 711, it is determined if a FRX response packet is received in response to the query. If not, control passes step 713. In step 713 it is determined whether the wait time is greater than a predetermined or dynamically determined excessive wait time, such as 5 minutes, or any time suitable for proper treatment of the patient, e.g., as determined by AI/ML. If not, then the wait time is not excessive; and control passes back to step 711. In some embodiments, if at least one response is received, or if the patient has been handed off to a different facility or responsible party, then no further wait time is considered excessive. If the wait time is greater than the excessive wait time value, then it is considered excessive and control passes to step 715. In some embodiments, if at least one known medication is missing and the patient has not yet been handed off, then wait time is still considered excessive even if a previous response was received. In step 715, the FRX query packet is sent again to the same or a backup FRX server. If the patient ID is known from a previous response, then the FRX query packet sent includes the patient ID, e.g., in patient information field 358. Control then returns to step 711.

If it is determined in step 711 that a FRX response packet (e.g., packet 500) is received, then control passes to step 721. In step 721 any locally saved patient and medication information is updated based on the information in the FRX response packet 500. In step 723, areas of the FRX response GUI 600 are populated or otherwise updated based on the information received in the FRX response packet 500.

In step 725, the FRX response GUI 600 is operated in response to navigation by a user to present requested information. Thus, a healthcare worker in communication with the user, such as a first responder, is enabled to properly treat the patient based on the presented information. For example, the healthcare worker is able to order and/or administer medication that the patient has missed but should be taking. Similarly, the healthcare worker avoids administering any medications that the patient should not receive, as known to the user due to information in the contra-indication area of the GUI 600. In some embodiments, step 725 includes handing off the patient to another responsible party, such as a facility to which the patient has been delivered. In some embodiments, this is done by indicating the handoff party link in a next party link field (not shown) in the response GUI 600 and activating a handoff button (not shown) in the response GUI 600. In response to the handoff button being activated, the client process sends one or more data packets 200, to the next party link, with data from the patient data structure 450, if any, in the local data structure 143, or sends a message to the server 182 that causes the server 182 to send one or more data packets 200, to the next party link, with data from the patient data structure 450 in the server data structure 142, or some combination.

In step 727 it is determined if end conditions are satisfied, such as the patient is stable in a facility, or the first responders have handed the patient over to another facility, or a new call is made for the first responders. If end conditions are not satisfied, control passes back to step 711 to receive any further updates from the FRX server in the form of another FRX response packet. Otherwise, the process ends.

FIG. 8 is a flow diagram that illustrates an example method for a FRX server module 182, according to an embodiment. In step 801, the server receives, and stores in FRX pharmacy data structure 400, information about the medication providers and any APIs each has for requesting prescription information. Any method may be used to receive this data, including having the data manually programmed into computer instructions, manually inputting the data into the data structure 450, receiving the data in response to a prompt to a user, or receiving the data from a remote host either unsolicited or in response to a query or a subscription, or using web crawl software and any associated AI/M. In some embodiments, step 801 includes assigning each unique medication provider a unique pharmacy ID value to store in field 412. In some embodiments, step 801 includes inserting into field 418 information related to translating fields used in the FRX system into parameter values for the particular pharmacy API, including the corresponding API parameter name or order in an argument list, units conversion, offsets scaling, compression, encryption, or authentication/ login credentials, or some combination. In some embodiments, such formatting is implemented to comply with all privacy requirements such as HIPAA and NIST.

In step 803, each pharmacy occupying a record in the pharmacy data structure is contacted, on a regular schedule or random basis, to ensure that the API is properly working and any information in the pharmacy data structure is still correct. If any problem is encountered, step 803 includes any automatic or manual intervention, or application of AI/ML, such as updates, to correct the problem.

In step 811 it is determined whether a FRX query packet 350 has been received from a FRX client 183. If not, control passes to step 871. In step 871, it is determined if end conditions are satisfied, such as powering off or going offline for service or updates. If so, then the process ends. Otherwise, control passes back to step 803, described above, to check the interactions with the pharmacy APIs 188.

If it is determined in step 811 that a FRX query packet 350 has been received, then control passes to step 813. In step 813, it is determined, based on the patient information in field 358, whether there is already a record for that patient in the FRX patient data structure 450 at the FRX server 182, in data structures 142. In some embodiments this is based on a FRX system generated unique patient ID that had been passed to the FRX client 183 in a previous response. If not, then control passes to step 815 in which a new patient record 460 is added to the FRX patient data structure 450 and a unique patient ID is generated and stored in field 462. In either case, control eventually passes to step 817.

In step 817, the fields in patient record 460 for the indicated patient are populated based on the information in the FRX query packet 350. For example, the time indicated in field 356 of the query is used to update the last update timestamp field 466. Any pharmacy information in the medication information field 360 is compared to the pharmacy record to determine the FRX unique pharmacy ID and the corresponding pharmacy ID is stored in field 479 for each medication from that pharmacy and also added to the pharmacy list field 468 for the patient. Control passes to step 821.

In step 821, it is determined whether there is a next listed pharmacy to contact. If so, control passes to step 823. If not, then control passes to step 831. If the query packet included any pharmacy information in the medication information fields 360, then that pharmacy is listed. If the query packet did not indicate any pharmacy, and no prior searches have found a pharmacy for this patient, then no pharmacy is listed. If all listed pharmacies have been contacted, then there is no further listed pharmacy to contact. If there is a listed pharmacy to contact, control passes to step 823.

In step 823, a request for patient prescription information is formatted for the API of the current pharmacy using the patient information available in patient record 460 for the current patient and the formatting information in pharmacy API format field 418 for the current pharmacy. A connection is made to the link in pharmacy link field 416 and the formatted patient information is provided to the API. For example, a pharmacy request message or data packet is sent to the API 188 of the corresponding pharmacy. In response, prescription information is returned by the API. For example, a pharmacy response message or data packet is received from the API 188 of the corresponding pharmacy.

In step 825, the patient and prescription information returned through the pharmacy API is reformatted for use by the FRX system using the formatting information in pharmacy API field 418. The resulting patient information and medication information is used to update any fields in the patient record 460 for the current patient. The last update timestamp field 465 is updated with the current time at the FRX server. In some embodiments, if the patient information was not complete, the pharmacy will send patient information for one or more patients that most closely match the patient information provided. Based on this information and any other information provided in the query, like prescriber or SSN, step 825 includes paring down the returned information to find the information pertinent to the most likely correct patient, using any probabilistic logic for disambiguation, including any AI/ML. For example AI/ML or other algorithms can use the prescription, prescriber, date and other information to eliminate duplicate or conflicting prescription returns from among multiple pharmacies. Control then passes back to step 821 to find the next listed pharmacy. In some embodiments in step 821, all APIs are queried for listed pharmacies even before every or any response is received from a previously queried pharmacy API.

When it is determined in step 821 that there are no more listed pharmacies to contact, control passes to step 831. In step 831, it is determined whether any patient or medication information has been updated, e.g., by checking the last update timestamp. If not, then control passes directly to step 841, described below. If so, then in step 833 the FRX response packet is populated and sent back to the FRX client 183, to be processed as described above with respect to FIG. 7. An advantage of this preemptive response is that it provides important information as quickly as possible to the user who may be a first responder in an emergency situation. Control then passes from step 833 to step 841. In some embodiments, steps 831 and 833 are skipped and no response packet is sent until previously unlisted medication providers are contacted for patient information based on region information, as in the following steps.

Whether steps 831 and 833 are skipped or not, control passes eventually to step 841. In step 841 it is determined whether there is a next pharmacy to check in one or more regions. It is anticipated that in some emergencies the user will not know any, or every, pharmacy that provides medication to the patient. To find previously unknown providers of medication to the patient, pharmacies are checked by region. Either or both of two natural regions can be established based on the information available to the users. The first region is the region in which the user is operating, e.g., the user' city, county, zip code, state (or province) or country, or some combination. This information has been provided in the FRX query message in field 354 based on the user's input at the user ID active area 301 of the query GUI 300. The second region is the region where the patient resides, which may be included in the patient information field 358 based on the user's input at the patient zip code active area 318. In addition, each pharmacy is associated with a service area indicated in the pharmacy record 410 by data held in pharmacy region field 414. Thus, in some embodiments, step 841 includes determining whether there is a pharmacy, not yet contacted, which has a service region that overlaps either the region of the user or the region of the patient's residence, or some combination. If so, then that pharmacy is contacted. In step 843 and 845 the API of that pharmacy is used to update the patient information or medication information—as already described above for steps 823 and 825, respectively. Control then passes back to step 841. In some embodiments in step 841, all APIs are queried for pharmacies that cover the region(s) even before every or any response is received from a previously queried pharmacy API.

When it is determined in step 841 that there is no other pharmacy to contact in either or both regions, control passes to step 851. In steps 851 and 853 it is determined if there has been an update to the patient record; and, if so an FRX response packet is sent, as already described above for steps 831 and 833, respectively. In some embodiments, if no pharmacy contacted has information about the patient, step 853 includes in field 570 a null value, thus indicating no medication information was found from any contacted medication providers. For example, fields corresponding to fields 471 through 477 contain null values, the field corresponding to fill region 476 holds data that indicates the largest region in which all pharmacies were contacted. Control then passes to step 861

In step 861, it is determined if either or both regions should be expanded. In some embodiments, this determination is made based on how many contacted medication providers have returned information about the current patient. In some embodiments, this decision is based on AI/ML. If that number is below some threshold, such as 1 or 2, then, at least one region is expanded by adding one or more adjacent regions or going to a larger region such as a state that overlaps the original region such as a zip code in that state. Then control passes back to step 841 to contact the next medication provider in the new region which has not already been contacted. In some embodiments in step 851, all APIs are queried for pharmacies not yet queried even before every or any response is received from a previously queried pharmacy API.

If it is determined in step 861 that the region is not being expanded, then control passes to step 871 to end the process, or to revisit the pharmacies in the pharmacy database to ensure the APIs are still functional, as described above.

The system 101 thus allows a person in the field, including medical and health professionals certified by the state to perform the appropriate level of care, or a person at a point of care, such as a hospital, to query multiple pharmacy databases practically instantly, but at least in real-time or near-real time, suitable for proper care of the patient and compile a list of the most recent medications dispensed. As used herein instantly is one second or less, near-real time is ten seconds or less, and near-real time is ten minutes or less. Some embodiments give a First Responder (EMT, Medic, etc.) in the field the ability to query pharmacies and point of care providers in a timely way to determine the last prescription drugs received by the patient. Because total perceived time is affected by network conditions such as congestion and link availability outside the control of system 101, functions within each of the FRX client 183 or FRX server 182 are advantageously configured to be performed instantly (one second or less) or in real-time (ten seconds or less) so that total perceived time is near real time (within ten minutes) or better.

### 3. Computational Hardware Overview

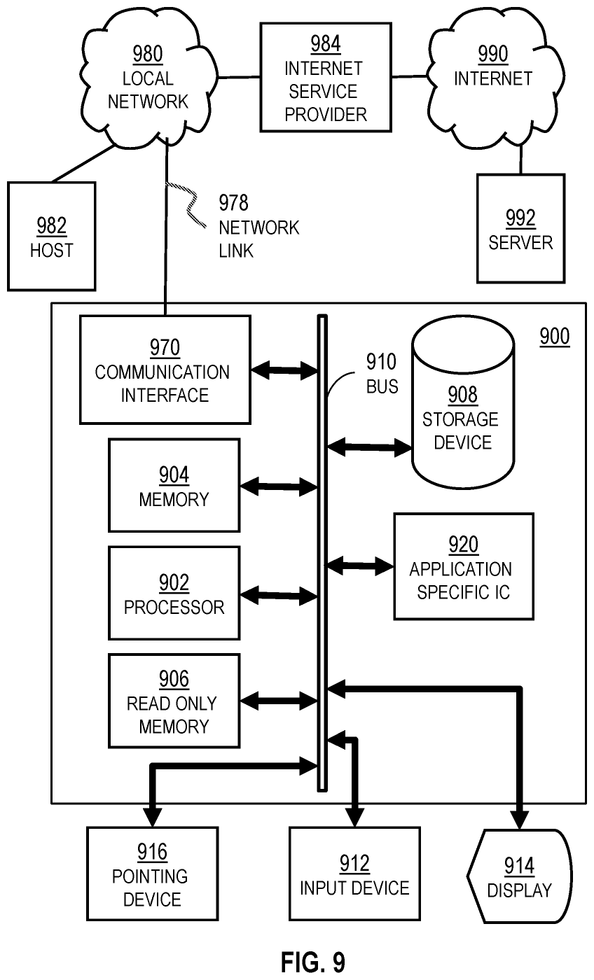

FIG. 9 is a block diagram that illustrates a computer system 900 upon which an embodiment of the invention may be implemented. Computer system 900 includes a communication mechanism such as a bus 910 for passing information between other internal and external components of the computer system 900. Information is represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit).). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range. Computer system 900, or a portion thereof, constitutes a means for performing one or more steps of one or more methods described herein.

A sequence of binary digits constitutes digital data that is used to represent a number or code for a character. A bus 910 includes many parallel conductors of information so that information is transferred quickly among devices coupled to the bus 910. One or more processors 902 for processing information are coupled with the bus 910. A processor 902 performs a set of operations on information. The set of operations include bringing information in from the bus 910 and placing information on the bus 910. The set of operations also typically include comparing two or more units of information, shifting positions of units of information, and combining two or more units of information, such as by addition or multiplication. A sequence of operations to be executed by the processor 902 constitutes computer instructions.

Computer system 900 also includes a memory 904 coupled to bus 910. The memory 904, such as a random access memory (RAM) or other dynamic storage device, stores information including computer instructions. Dynamic memory allows information stored therein to be changed by the computer system 900. RAM allows a unit of information stored at a location called a memory address to be stored and retrieved independently of information at neighboring addresses. The memory 904 is also used by the processor 902 to store temporary values during execution of computer instructions. The computer system 900 also includes a read only memory (ROM) 906 or other static storage device coupled to the bus 910 for storing static information, including instructions, that is not changed by the computer system 900. Also coupled to bus 910 is a non-volatile (persistent) storage device 908, such as a magnetic disk or optical disk, for storing information, including instructions, that persists even when the computer system 900 is turned off or otherwise loses power.

Information, including instructions, is provided to the bus 910 for use by the processor from an external input device 912, such as a keyboard containing alphanumeric keys operated by a human user, or a sensor. A sensor detects conditions in its vicinity and transforms those detections into signals compatible with the signals used to represent information in computer system 900. Other external devices coupled to bus 910, used primarily for interacting with humans, include a display device 914, such as a cathode ray tube (CRT) or a liquid crystal display (LCD), for presenting images, and a pointing device 916, such as a mouse or a trackball or cursor direction keys, for controlling a position of a small cursor image presented on the display 914 and issuing commands associated with graphical elements presented on the display 914.

In the illustrated embodiment, special purpose hardware, such as an application specific integrated circuit (IC) 920, is coupled to bus 910. The special purpose hardware is configured to perform operations not performed by processor 902 quickly enough for special purposes. Examples of application specific ICs include graphics accelerator cards for generating images for display 914, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 900 also includes one or more instances of a communications interface 970 coupled to bus 910. Communication interface 970 provides a two-way communication coupling to a variety of external devices that operate with their own processors, such as printers, scanners and external disks. In general the coupling is with a network link 978 that is connected to a local network 980 to which a variety of external devices with their own processors are connected. For example, communication interface 970 may be a parallel port or a serial port or a universal serial bus (USB) port on a personal computer. In some embodiments, communications interface 970 is an integrated services digital network (ISDN) card or a digital subscriber line (DSL) card or a telephone modem that provides an information communication connection to a corresponding type of telephone line. In some embodiments, a communication interface 970 is a cable modem that converts signals on bus 910 into signals for a communication connection over a coaxial cable or into optical signals for a communication connection over a fiber optic cable. As another example, communications interface 970 may be a local area network (LAN) card to provide a data communication connection to a compatible LAN, such as Ethernet. Wireless links may also be implemented. Carrier waves, such as acoustic waves and electromagnetic waves, including radio, optical and infrared waves travel through space without wires or cables. Signals include man-made variations in amplitude, frequency, phase, polarization or other physical properties of carrier waves. For wireless links, the communications interface 970 sends and receives electrical, acoustic or electromagnetic signals, including infrared and optical signals, that carry information streams, such as digital data.

The term computer-readable medium is used herein to refer to any medium that participates in providing information to processor 902, including instructions for execution. Such a medium may take many forms, including, but not limited to, non-volatile media, volatile media and transmission media. Non-volatile media include, for example, optical or magnetic disks, such as storage device 908. Volatile media include, for example, dynamic memory 904. Transmission media include, for example, coaxial cables, copper wire, fiber optic cables, and waves that travel through space without wires or cables, such as acoustic waves and electromagnetic waves, including radio, optical and infrared waves. The term computer-readable storage medium is used herein to refer to any medium that participates in providing information to processor 902, except for transmission media.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read. The term non-transitory computer-readable storage medium is used herein to refer to any medium that participates in providing information to processor 902, except for carrier waves and other signals.

Logic encoded in one or more tangible media includes one or both of processor instructions on a computer-readable storage media and special purpose hardware, such as ASIC 920.

Network link 978 typically provides information communication through one or more networks to other devices that use or process the information. For example, network link 978 may provide a connection through local network 980 to a host computer 982 or to equipment 984 operated by an Internet Service Provider (ISP). ISP equipment 984 in turn provides data communication services through the public, world-wide packet-switching communication network of networks now commonly referred to as the Internet 990. A computer called a server 992 connected to the Internet provides a service in response to information received over the Internet. For example, server 992 provides information representing video data for presentation at display 914.

The invention is related to the use of computer system 900 for implementing the techniques described herein. According to one embodiment of the invention, those techniques are performed by computer system 900 in response to processor 902 executing one or more sequences of one or more instructions contained in memory 904. Such instructions, also called software and program code, may be read into memory 904 from another computer-readable medium such as storage device 908. Execution of the sequences of instructions contained in memory 904 causes processor 902 to perform the method steps described herein. In alternative embodiments, hardware, such as application specific integrated circuit 920, may be used in place of or in combination with software to implement the invention. Thus, embodiments of the invention are not limited to any specific combination of hardware and software.

The signals transmitted over network link 978 and other networks through communications interface 970, carry information to and from computer system 900. Computer system 900 can send and receive information, including program code, through the networks 980, 990 among others, through network link 978 and communications interface 970. In an example using the Internet 990, a server 992 transmits program code for a particular application, requested by a message sent from computer 900, through Internet 990, ISP equipment 984, local network 980 and communications interface 970. The received code may be executed by processor 902 as it is received, or may be stored in storage device 908 or other non-volatile storage for later execution, or both. In this manner, computer system 900 may obtain application program code in the form of a signal on a carrier wave.

Various forms of computer readable media may be involved in carrying one or more sequences of instructions or data or both to processor 902 for execution. For example, instructions and data may initially be carried on a magnetic disk of a remote computer such as host 982. The remote computer loads the instructions and data into its dynamic memory and sends the instructions and data over a telephone line using a modem. A modem local to the computer system 900 receives the instructions and data on a telephone line and uses an infra-red transmitter to convert the instructions and data to a signal on an infra-red carrier wave serving as the network link 978. An infrared detector serving as communications interface 970 receives the instructions and data carried in the infrared signal and places information representing the instructions and data onto bus 910. Bus 910 carries the information to memory 904 from which processor 902 retrieves and executes the instructions using some of the data sent with the instructions. The instructions and data received in memory 904 may optionally be stored on storage device 908, either before or after execution by the processor 902.

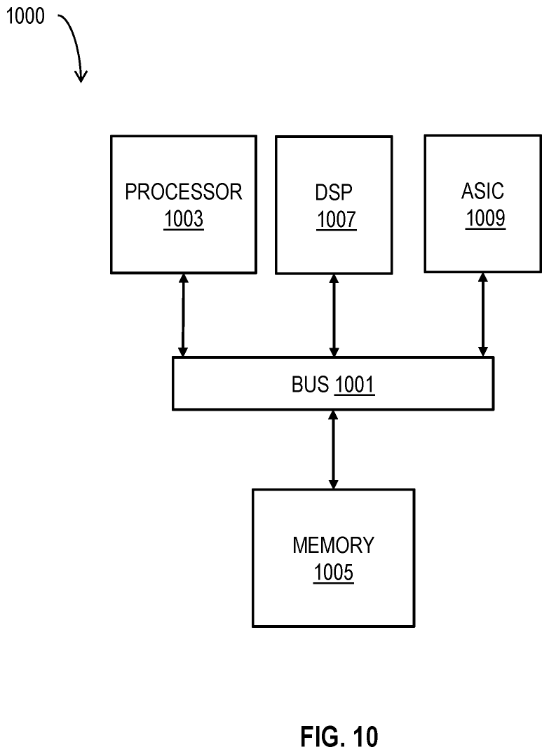

FIG. 10 illustrates a chip set 1000 upon which an embodiment of the invention may be implemented. Chip set 1000 is programmed to perform one or more steps of a method described herein and includes, for instance, the processor and memory components described with respect to FIG. 9 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip. Chip set 1000, or a portion thereof, constitutes a means for performing one or more steps of a method described herein.

In one embodiment, the chip set 1000 includes a communication mechanism such as a bus 1001 for passing information among the components of the chip set 1000. A processor 1003 has connectivity to the bus 1001 to execute instructions and process information stored in, for example, a memory 1005. The processor 1003 may include one or more processing cores with each core configured to perform independently. A multi-core processor enables multiprocessing within a single physical package. Examples of a multi-core processor include two, four, eight, or greater numbers of processing cores. Alternatively or in addition, the processor 1003 may include one or more microprocessors configured in tandem via the bus 1001 to enable independent execution of instructions, pipelining, and multithreading. The processor 1003 may also be accompanied with one or more specialized components to perform certain processing functions and tasks such as one or more digital signal processors (DSP) 1007, or one or more application-specific integrated circuits (ASIC) 1009. A DSP 1007 typically is configured to process real-world signals (e.g., sound) in real time independently of the processor 1003. Similarly, an ASIC 1009 can be configured to perform specialized functions not easily performed by a general purpose processor. Other specialized components to aid in performing the inventive functions described herein include one or more field programmable gate arrays (FPGA) (not shown), one or more controllers (not shown), or one or more other special-purpose computer chips.

The processor 1003 and accompanying components have connectivity to the memory 1005 via the bus 1001. The memory 1005 includes both dynamic memory (e.g., RAM, magnetic disk, writable optical disk, etc.) and static memory (e.g., ROM, CD-ROM, etc.) for storing executable instructions that when executed perform one or more steps of a method described herein. The memory 1005 also stores the data associated with or generated by the execution of one or more steps of the methods described herein.

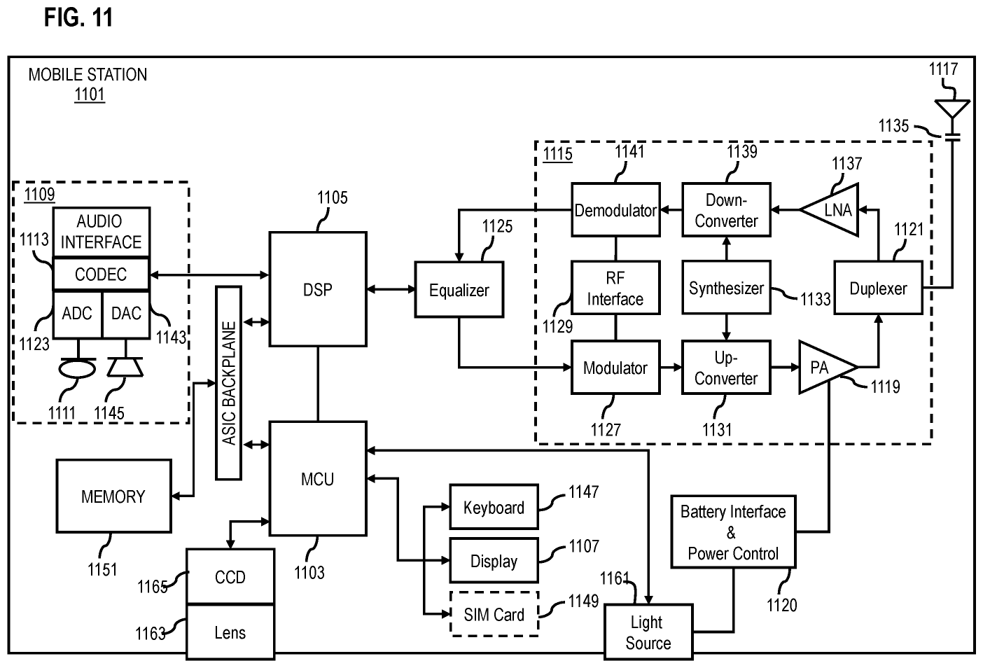

FIG. 11 is a diagram of example components of a mobile terminal 1100 (e.g., cell phone handset) for communications, which is capable of operating in the system of FIG. 1A, according to one embodiment. In some embodiments, mobile terminal 1101, or a portion thereof, constitutes a means for performing one or more steps described herein. Generally, a radio receiver is often defined in terms of front-end and back-end characteristics. The front-end of the receiver encompasses all of the Radio Frequency (RF) circuitry whereas the back-end encompasses all of the base-band processing circuitry. As used in this application, the term “circuitry” refers to both: (1) hardware-only implementations (such as implementations in only analog and/or digital circuitry), and (2) to combinations of circuitry and software (and/or firmware) (such as, if applicable to the particular context, to a combination of processor(s), including digital signal processor(s), software, and memory(ies) that work together to cause an apparatus, such as a mobile phone or server, to perform various functions). This definition of “circuitry” applies to all uses of this term in this application, including in any claims. As a further example, as used in this application and if applicable to the particular context, the term “circuitry” would also cover an implementation of merely a processor (or multiple processors) and its (or their) accompanying software/or firmware. The term “circuitry” would also cover if applicable to the particular context, for example, a baseband integrated circuit or applications processor integrated circuit in a mobile phone or a similar integrated circuit in a cellular network device or other network devices.

Pertinent internal components of the telephone include a Main Control Unit (MCU) 1103, a Digital Signal Processor (DSP) 1105, and a receiver/transmitter unit including a microphone gain control unit and a speaker gain control unit. A main display unit 1107 provides a display to the user in support of various applications and mobile terminal functions that perform or support the steps as described herein. The display 1107 includes display circuitry configured to display at least a portion of a user interface of the mobile terminal (e.g., mobile telephone). Additionally, the display 1107 and display circuitry are configured to facilitate user control of at least some functions of the mobile terminal. An audio function circuitry 1109 includes a microphone 1111 and microphone amplifier that amplifies the speech signal output from the microphone 1111. The amplified speech signal output from the microphone 1111 is fed to a coder/decoder (CODEC) 1113.

A radio section 1115 amplifies power and converts frequency in order to communicate with a base station, which is included in a mobile communication system, via antenna 1117. The power amplifier (PA) 1119 and the transmitter/modulation circuitry are operationally responsive to the MCU 1103, with an output from the PA 1119 coupled to the duplexer 1121 or circulator or antenna switch, as known in the art. The PA 1119 also couples to a battery interface and power control unit 1120.

In use, a user of mobile terminal 1101 speaks into the microphone 1111 and his or her voice along with any detected background noise is converted into an analog voltage. The analog voltage is then converted into a digital signal through the Analog to Digital Converter (ADC) 1123. The control unit 1103 routes the digital signal into the DSP 1105 for processing therein, such as speech encoding, channel encoding, encrypting, and interleaving. In one embodiment, the processed voice signals are encoded, by units not separately shown, using a cellular transmission protocol such as enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., microwave access (WiMAX), Long Term Evolution (LTE) networks, code division multiple access (CDMA), wideband code division multiple access (WCDMA), wireless fidelity (WiFi), satellite, and the like, or any combination thereof.

The encoded signals are then routed to an equalizer 1125 for compensation of any frequency-dependent impairments that occur during transmission through the air such as phase and amplitude distortion. After equalizing the bit stream, the modulator 1127 combines the signal with a RF signal generated in the RF interface 1129. The modulator 1127 generates a sine wave by way of frequency or phase modulation. In order to prepare the signal for transmission, an up- converter 1131 combines the sine wave output from the modulator 1127 with another sine wave generated by a synthesizer 1133 to achieve the desired frequency of transmission. The signal is then sent through a PA 1119 to increase the signal to an appropriate power level. In practical systems, the PA 1119 acts as a variable gain amplifier whose gain is controlled by the DSP 1105 from information received from a network base station. The signal is then filtered within the duplexer 1121 and optionally sent to an antenna coupler 1135 to match impedances to provide maximum power transfer. Finally, the signal is transmitted via antenna 1117 to a local base station. An automatic gain control (AGC) can be supplied to control the gain of the final stages of the receiver. The signals may be forwarded from there to a remote telephone which may be another cellular telephone, any other mobile phone or a land-line connected to a Public Switched Telephone Network (PSTN), or other telephony networks.

Voice signals transmitted to the mobile terminal 1101 are received via antenna 1117 and immediately amplified by a low noise amplifier (LNA) 1137. A down-converter 1139 lowers the carrier frequency while the demodulator 1141 strips away the RF leaving only a digital bit stream. The signal then goes through the equalizer 1125 and is processed by the DSP 1105. A Digital to Analog Converter (DAC) 1143 converts the signal and the resulting output is transmitted to the user through the speaker 1145, all under control of a Main Control Unit (MCU) 1103 which can be implemented as a Central Processing Unit (CPU) (not shown).

The MCU 1103 receives various signals including input signals from the keyboard 1147. The keyboard 1147 and/or the MCU 1103 in combination with other user input components (e.g., the microphone 1111) comprise a user interface circuitry for managing user input. The MCU 1103 runs a user interface software to facilitate user control of at least some functions of the mobile terminal 1101 as described herein. The MCU 1103 also delivers a display command and a switch command to the display 1107 and to the speech output switching controller, respectively. Further, the MCU 1103 exchanges information with the DSP 1105 and can access an optionally incorporated SIM card 1149 and a memory 1151. In addition, the MCU 1103 executes various control functions required of the terminal. The DSP 1105 may, depending upon the implementation, perform any of a variety of conventional digital processing functions on the voice signals. Additionally, DSP 1105 determines the background noise level of the local environment from the signals detected by microphone 1111 and sets the gain of microphone 1111 to a level selected to compensate for the natural tendency of the user of the mobile terminal 1101.

The CODEC 1113 includes the ADC 1123 and DAC 1143. The memory 1151 stores various data including call-incoming tone data and is capable of storing other data including music data received via, e.g., the global Internet. The software module could reside in RAM memory, flash memory, registers, or any other form of writable storage medium known in the art. The memory device 1151 may be, but not limited to, a single memory, CD, DVD, ROM, RAM, EEPROM, optical storage, magnetic disk storage, flash memory storage, or any other non-volatile storage medium capable of storing digital data.

An optionally incorporated SIM card 1149 carries, for instance, important information, such as the cellular phone number, the carrier supplying service, subscription details, and security information. The SIM card 1149 serves primarily to identify the mobile terminal 1101 on a radio network. The card 1149 also contains a memory for storing a personal telephone number registry, text messages, and user specific mobile terminal settings.

In some embodiments, the mobile terminal 1101 includes a digital camera comprising an array of optical detectors, such as charge coupled device (CCD) array 1165. The output of the array is image data that is transferred to the MCU for further processing or storage in the memory 1151 or both. In the illustrated embodiment, the light impinges on the optical array through a lens 1163, such as a pin-hole lens or a material lens made of an optical grade glass or plastic material. In the illustrated embodiment, the mobile terminal 1101 includes a light source 1161, such as a LED to illuminate a subject for capture by the optical array, e.g., CCD 1165. The light source is powered by the battery interface and power control module 1120 and controlled by the MCU 1103 based on instructions stored or loaded into the MCU 1103.

### 4. Alternatives, Deviations and Modifications

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus, a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about ×” implies a value in the range from 0.5× to 2×, for example, about 100 implies a value in a range from 50 to 200. Moreover, all ranges disclosed herein are to be understood to encompass any and all sub-ranges subsumed therein. For example, a range of “less than 10” for a positive only parameter can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

What is claimed is:

1. An automatic method to provide timely medication information, comprising:
storing in a first database, for each of a plurality of different medication providers, a pharmacy ID field holding data that uniquely indicates the medication provider, a pharmacy link field holding data that indicates a communications network link for the medication provider, and a pharmacy API format field holding data that indicates an application programming interface format for requesting patient medication information and for receiving patient medication information from the medication provider; and
in response to receiving over a network a first client query packet from a client process on a remote network node, wherein the client query packet includes a patient field that holds data that indicates patent information for a first patient,
selecting a first medication provider of the plurality of medication providers,
formatting a first pharmacy request packet to request medication information for the first patient based on the API format field for the first medication provider, and
sending the first pharmacy request packet over the network to the communication network link in the pharmacy link field for the first medication provider.

2. The method as recited in claim 1, further comprising, storing data that indicates information in the first client query packet in a first patient data record.

3. The method as recited in claim 1, further comprising, in response to receiving over the network a first pharmacy response packet in response to the first pharmacy request packet, the steps of:
determining whether the first pharmacy response packet indicates the first patient is not among the records at the first medication provider;
if the first pharmacy response indicates the first patient is not among the records of the first medication provider, then
selecting a different second medication provider of the plurality of medication providers,
formatting a second pharmacy request packet to request medication information for the first patient based on the API format field for the second medication provider, and
sending the second pharmacy request packet over the network to the communication network link in the pharmacy link field for the second medication provider.

4. The method as recited in claim 1, further comprising, in response to receiving over the network a first pharmacy response packet in response to the first pharmacy request packet, the steps of:
retrieving first medication information for the first patient from the first pharmacy response packet based on the API format field for the first medication provider;
generating a first client response packet that include the first medication information for the first patient; and
sending the first client response packet over the network to the client process on the remote network node, where data is presented to a user based on the first medication information to enable the user to treat the first patent.

5. The method as recited in claim 4, further comprising, storing in a first patient data record data that indicates information in the first pharmacy response packet.

6. The method as recited in claim 5, further comprising, in response to receiving, over the network from a second remote network node, a second client query packet indicating the first patient,
generating a second client response packet that includes the first medication information for the first patient; and
sending the second client response packet over the network to the client process on the second remote network node.

7. The method as recited in claim 2, further comprising, in response to receiving over the network a first pharmacy response packet in response to the first pharmacy request packet, the steps of:
retrieving first medication information and independent patient information for the first patient from the first pharmacy response packet based on the API format field for the first medication provider;
determining whether the independent patient information is consistent with data already in a first patient data record; and
when the independent patient information is consistent ,then
storing the first medication information and any new independent patient information in the patient data record,
generating a first client response packet that include the first medication information for the first patient, and
sending the first client response packet over the network to the client process on the remote network node, where data is presented to a user based on the first medication information to enable the user to treat the first patient.

8. The method as recited in claim 1, wherein:
said storing in the first database further comprises storing in the first database, for each of the plurality of different medication providers, a pharmacy region field holding data that indicates a geographic region served by the medication provider; and
the client query packet includes a region field that holds data that indicates a geographic region applicable for the first patient; and
said selecting the first medication provider of the plurality of medication providers further comprises selecting the first medication provider based at least in part on proximity between the geographic region served by the medication provider and the geographic region applicable for first patient.

9. The method as recited in claim 8, wherein the geographical region applicable for the first patient is a geographical region that includes a home address for the first patient.

10. The method as recited in claim 8, wherein the geographical region applicable for the first patient is a geographical region in a vicinity of the remote network node.

11. The method as recited in claim 8, wherein said selecting the first medication provider based at least in part on proximity between the geographic region served by the medication provider and the geographic region applicable for first patient further comprises geographically expanding a geographic region included in the first pharmacy request packet until a first pharmacy response packet that includes first medication for the first patient is returned in response to sending the first pharmacy request packet.

12. The method as recited in claim 1, wherein:
the client query packet includes a pharmacy field that holds data that indicates a second medication provider applicable for first patient; and
said selecting the first medication provider of the plurality of medication providers further comprises selecting the first medication provider based on the second medication provider applicable for first patient.

13. An automatic method implemented on a processor to provide timely medication information, comprising:
presenting on a display device a graphical user interface (GUI) for a user, wherein the GUI is configured to allow the user to enter what information is available to the user about a patient;
generating a first client query packet that includes a patient field that holds data that indicates patent information for a first patient based on user input to the GUI;
sending the first client query packet to a remote server that is configured to request medication information for the first patient based on an API format field for a first medication provider.

14. The method as recited in claim 13, wherein the information for the first client query packet is provided at least in part by optical character recognition of a document scanned by the user on site.

15. The method as recited in claim 13, further comprising:
in response to sending the first client query packet to the remote server, receiving from the remote server a first client response packet that includes data that indicates first medication information for the first patient; and
in response to receiving the first client response packet, presenting on the display device the first medication information for the first patient.

16. The method as recited in claim 15, wherein the GUI is configured to present the first medication information for the first patient.

17. A non-transitory computer-readable medium carrying one or more sequences of instructions, wherein execution of the one or more sequences of instructions by one or more processors causes the one or more processors to perform the steps of claim 1.

18. A non-transitory computer-readable medium carrying one or more sequences of instructions, wherein execution of the one or more sequences of instructions by one or more processors causes the one or more processors to perform the steps of claim 13.

19. A system comprising:
at least one processor;
a network card for sending and receiving packets; and
at least one memory including one or more sequences of instructions,
the at least one memory and the one or more sequences of instructions configured to, with the at least one processor, cause the system to perform the steps of claim 1.

20. A system comprising:
at least one processor;
a display device;
a network card for sending and receiving packets; and
at least one memory including one or more sequences of instructions,
the at least one memory and the one or more sequences of instructions configured to, with the at least one processor, cause the system to perform the steps of claim 13.

21. A medication GUI presented on a device, comprising:
a first active area configured to receive patient information;
a second active area configured to receive medication information;
a third active area configured to receive location information;
a submit button configured upon activation to cause the device to send a query to a server that is configured upon receipt of the query to check medication information for the patient over at least one of a plurality of network links for a corresponding plurality of medication providers; and
a display area configured to present medication information corresponding to the patient information from at least one of the plurality of medication providers.
